(19) United States
(12) Patent Application Publication
(10) Pub. No.: US 2006/0263299 A1
(43) Pub. Date: Nov. 23, 2006

(54) Effect of substances on sexual function

(76) Inventors: Stacey Jill Sukoff Rizzo, Levittown, PA (US); Lee Erwin Schechter, Toms River, NJ (US); Sharon Rosenzweig-Lipson, East Brunswick, NJ (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/415,437

(22) Filed: May 2, 2006

Related U.S. Application Data
(60) Provisional application No. 60/682,379, filed on May 19, 2005.

(51) Int. Cl. A61K 49/00

(52) U.S. Cl. 424/9.2

(57) ABSTRACT

A preclinical animal model and method of using same to evaluate the effects of various substances (or quantities of substances), such as antidepressants, on sexual function in humans and other mammals is disclosed. Also disclosed are compositions comprising the substances evaluated by the present invention and method for using same to treat and/or prevent sexual dysfunction (or improve sexual function), as well as an apparatus used in the preclinical evaluation of the effects of substances on sexual function in humans and other mammals.

### PRIORITY DATA

This application claims priority under 35 U.S.C. §119 to Provisional Patent Application No. 60/682,379 filed on May 19, 2005.

### FIELD OF THE INVENTION

The present invention provides a method for evaluating the effects of various substances (or quantities of substances), such as antidepressants, on sexual function in mammals. In particular, the present invention provides an animal model for evaluating the effects of substances (or quantities of substances) on sexual function. Also provided by the present invention is an apparatus used in the preclinical evaluation of the effects of substances on sexual function. Further, the present invention provides substances (compounds, etc.), and compositions comprising the substances (pharmaceutical compositions, etc.), for preventing and/or treating sexual dysfunction, as well as improving sexual function.

### BACKGROUND OF THE INVENTION

Sexual dysfunction is defined as a disruption in any part of the sexual cycle, inclusive of disorders of desire, arousal, orgasm, and resolution (Diagnostic and Statistical Manual of Mental Disorders IV, 1994). Sexual dysfunction is a significant clinical problem which can affect both males and females. The causes of sexual dysfunction may be both organic as well as psychological. Organic aspects of sexual dysfunction are typically caused by underlying vascular diseases, such as those associated with hypertension or diabetes mellitus, by prescription medication and/or by psychiatric disease such as depression. Physiological factors include fear, performance anxiety and interpersonal conflict. Sexual dysfunction impairs sexual performance, diminishes self-esteem and disrupts personal relationships thereby inducing personal distress. In the clinic, sexual dysfunction disorders have been divided into female sexual dysfunction disorders and male sexual dysfunction disorders (Melman et al 1999).

Sexual dysfunction is considered a symptomology of clinical depression (Angst et al., 1998; Montgomery et al., 2002) and is also associated with drug treatment, such as antidepressant treatment (Gregorian et al., 2002; Ferguson, 2001). For example, although antidepressant therapies may restore sexual functioning in clinically depressed patients, this is merely a temporary remedy; a staggering 20-45% of antidepressant-treated patients report disruptions in sexual functioning as a side-effect of their treatment (Gregorian et al., 2002; Clayton et al., 2002). Antidepressant-induced sexual dysfunction has been reported across all classes of antidepressants (Montgomery et al., 2002).

One of the first studies evaluating the effects of antidepressants on sexual function revealed that 30% of patients treated with the tricyclic antidepressant imipramine reported impairment in sexual function (Harrison et al., 1986). More recently, a number of clinical studies have focused on selective serotonin reuptake inhibitors (SSRIs), such as fluoxetine and the dopamine, and norepinephrine reuptake inhibitor, bupropion and have reported sexual dysfunction at rates of 73% and 14%, respectively (Worthington et al., 2003; Modell et al., 1997). It has been suggested that onset of disruptions in sexual functioning can occur as early as 7 days after initiation of antidepressant treatment (Ferguson 2001; Croft et al., 1999). These side-effects are notorious for resulting in poor patient compliance as it has been reported that less than 30% of patients complete the recommended 6-9 month treatment regimen following a single acute episode of an adverse sexual event (Nurnberg, 2001).

A number of strategies have been attempted by clinicians to alleviate the sexual dysfunction associated with antidepressant treatments. Recently, concomitant medications have been used to offset the sexual side effects of antidepressants and have succeeded with varying efficacy (Fava and Rankin, 2002; Brill, 2004; Hirschfeld, 2003). These adjunct pharmacotherapies have included among others, the alpha-adrenergic receptor antagonist yohimbine (Price and Gunhaus, 1990), the phosphodiesterase type 5 inhibitor sildenafil (Nurnberg et al., 2003; Fava et al., 1998), and the dopamine agonists methylphenidate (Bartlik and Kaplan, 1995) and ropinirole (Worthington et al., 2002).

Previously, development of predictive and efficient preclinical animal models of sexual dysfunction have proven elusive. Methods which evaluate erectile response such as contractile blood vessel response and pharmacologically induced penile erections studies are not an interpretation of sexual behavior per se, but rather a method for evaluating erectile response. Paired-mating studies are extremely low-throughput, labor-intensive, and require large animal numbers (Olivier, et al; 2001). This type of methodology also requires training of sexual behavior over multiple sessions and weeks to determine stable baselines of sexual activity and selecting for a population of high performers.

Accordingly, there is a need for a predictive and efficient method for evaluating the effects of substances, such as pharmaceuticals, e.g., antidepressants, on sexual function in humans and other mammals. In particular, there is a need for a predictive and efficient animal model of sexual function. Further, there is a need for methods for evaluating antidepressant-induced sexual dysfunction and potential treatments for antidepressant-induced sexual dysfunction, as well as a need for compounds and compositions identified by the methods described herein.

### BRIEF DESCRIPTION OF THE FIGURES

FIG. 1 is a graph illustrating results of a study to evaluate the effect of sexual experience versus sexual inexperience in the presence or absence of female stimuli on non-contact penile erections in male Sprague-Dawley rats.

FIG. 2 is a graph illustrating the results of a study to evaluate the effects of vehicle, fluoxetine (10 mg/kg/day intraperitoneally (i.p.)), desipramine (10 mg/kg/day, i.p.), and bupropion (20 mg/kg/day, i.p.) treatment after 14-day treatment on the mean number of non-contact penile erections of sexually-experienced male rats in a 30-min test session in the presence of a receptive female.

FIG. 3 is a graph illustrating the results of a study to evaluate the effects of vehicle or fluoxetine (10 mg/kg/day, i.p.) following acute, subchronic, and chronic treatment on the number of non-contact penile erections in sexually-experienced males in 30-min test sessions in the presence of receptive females.

FIG. 4 is a graph illustrating the results of a study to evaluate the effects of vehicle or desipramine (10 mg/kg/day, i.p.) following acute, subchronic, and chronic treatment on the number of non-contact penile erections in sexually-experienced males in 30 min test sessions in the presence of receptive females.

FIG. 5 is a graph illustrating the results of a study to evaluate the effects of vehicle or bupropion (20 mg/kg/day, i.p.) following acute, subchronic, and chronic treatment on the number of non-contact penile erections in sexually-experienced males in 30 min test sessions in the presence of receptive females.

FIG. 6 is a graph illustrating the results of a study to evaluate the effects of acute apomorphine treatment (0, 0.03, or 0.1 mg/kg, subcutaneous (s.c.)) co-administered with chronic (14 day) fluoxetine (10 mg/kg/day, i.p.) on the number of non-contact penile erections in sexually-experienced males in 30 min test sessions in the presence of receptive females.

FIG. 7 is a graph illustrating the results of a study to evaluate the effects of treatment with sildenafil citrate (available as VIAGRA from Pfizer Inc. of New York, N.Y.), yohimbine and dopamine agonists co-administered with fluoxetine (over 14 days) on the number of non-contact penile erections in sexually-experienced males.

### SUMMARY OF THE INVENTION

The present invention provides a method for evaluating the effects of substances (and different quantities of substances), such as pharmaceuticals, e.g., antidepressants, on sexual function in humans and other mammals. The present invention also provides a predictive and efficient animal model of sexual function. Further, the present invention provides methods for evaluating antidepressant-induced sexual dysfunction and potential treatments for antidepressant-induced sexual dysfunction. Compounds and compositions identified by the methods described herein are also provided by the methods of the present invention.

According to an embodiment, the present invention provides a method for evaluating the effect of a pharmaceutical composition on the sexual function of a male mammal, the method comprising: (a) administering a pharmaceutical composition to one or more male mammals, (b) placing the one or more male mammals in a testing arena comprising one or more female mammals, wherein the males are separated from the female by a separating means (barrier) which prevents physical contact between the male and female mammals, wherein the separating means does not inhibit or prevent the exchange of olfactory, auditory or visual stimuli between the male and females, and (c) recording the number of observed penile erections in the one or more male mammals; wherein a change in the number of penile erections, relative to control male mammals receiving a placebo composition, indicates that the pharmaceutical composition has altered the sexual function of the one or more male mammals.

According to a further embodiment, the present invention provides a method for evaluating the effect of a pharmaceutical composition on the alleviation or reversal of a sexual dysfunction in a male mammal, the method comprising the steps of: (a) administering a first pharmaceutical composition which induces sexual dysfunction to one or more male mammals, (b) placing the one or more male mammals of step (a) in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females, (c) recording the number of observed penile erections in the male mammals of step (b), wherein a decrease in the number of penile erections, relative to control males receiving a placebo composition, indicates that the first pharmaceutical composition has induced a sexual dysfunction in the male mammal, (d) administering a second pharmaceutical composition to the same male mammals used in step (a), (e) placing the one or more male mammals of step (d) in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females, and (f) recording the number of observed penile erections in the male mammals of step (e); wherein an increase in the number of penile erections, relative to recordings of step (c), indicates that the second pharmaceutical composition reverses the sexual dysfunction induced by the first pharmaceutical composition.

According to an even further embodiment, the present invention provides a pharmaceutical composition in an amount sufficient to provide an antidepressant effect without causing sexual dysfunction when administered to a human subject, wherein the amount of the pharmaceutical composition is determined according to the methods described herein.

According to another embodiment of the present invention, the present invention provides a method of determining the amount of a pharmaceutical composition to administer to a patient to prevent impairment of sexual function or to improve sexual function comprising: monitoring the quantity of penile erections in male animals that are administered the amount of the agent and then placed in a testing arena that exposes the male animals to female animals through a barrier that prevents physical contact between the male animals and any female animal while not preventing the male animals from seeing, smelling or hearing a female animal.

According to yet another embodiment, the present invention provides an apparatus for testing the effect of a composition on sexual function in a mammal comprising: a separating means which prevents physical contact between the male and female mammals, wherein the separating means does not inhibit or prevent the exchange of olfactory, auditory or visual stimuli between the male and females.

### DETAILED DESCRIPTION OF THE INVENTION

The inventors have discovered a novel animal model which is unexpectedly predictive and efficient in evaluating the effect of substances (or different quantities of substances) on sexual function in humans and other mammals. A novel animal model incorporating rats was studied. It was unexpectedly discovered that the rat model patterned reports of sexual dysfunction in the clinic associated with different antidepressant treatments as well as a time course for the onset of sexual dysfunction and the ability of adjunct treatment to reverse the sexual impairments produced by antidepressants. Thus, according to an embodiment, the present invention provides a method for evaluating the effects of substances on antidepressant-induced sexual dysfunction.

The time required to collect data using the method described herein is less than 21 days relative to the 3-6 month period inclusive of training sessions, required by other models (e.g., paired mating models). Therefore, the method of the present invention is unexpectedly more efficient in assessing antidepressant-induced sexual dysfunction than previously known models.

In the studies described herein, male Sprague-Dawley rats were allowed access to sexually-receptive females during a single overnight mating session and then treated with antidepressants known to produce differing levels of sexual dysfunction clinically. At the conclusion of the antidepressant treatment period, the rats were observed for penile erections in the presence of sexually receptive females that were not accessible for contact, but served as visual, auditory, and olfactory stimuli in the testing area. Following 14 days of treatment, the selective serotonin re-uptake inhibitor (SSRI), fluoxetine (10 mg/kg), the tricyclic antidepressant, desipramine (10 mg/kg), and the dopamine and norepinephrine reuptake inhibitor, bupropion (20 mg/kg), reduced the number of penile erections, 9%, 43%, and 72%; respectively, relative to vehicle treated animals following 14 days of treatment. This rank order of the compounds propensity for reducing the number of penile erections is comparable to the rank order of the compounds' ability to produce sexual dysfunction clinically.

Like the delayed onset of antidepressant-induced sexual dysfunction clinically, there was a delayed onset of sexual deficits in this model. Fluoxetine did not affect penile erections following acute treatment, but produced a time-dependent reduction in the number of penile erections. Desipramine treatment resulted in a reduction in the number penile erections acutely and consistently over time. Acute, but not subchronic or chronic bupropion treatment resulted in a significant reduction in the number of penile erections. Additionally, acute treatment with the dopamine agonist apomorphine produced a dose-dependent reversal of the deficits in the number of non-contact penile erections produced by chronic fluoxetine treatment. Taken together, these data are consistent with the clinical deficits associated with antidepressants and is predictive of antidepressant-induced sexual dysfunction.

As used herein, sexual dysfunction was defined by the absence or reduction of penile erections compared to the consistent occurrence of non-contact penile erections resulting from sexually experienced vehicle treated animals introduced into a novel environment and in the presence of receptive females. This confirms previous reports (Sachs et al., 1994; Melis et al., 1998) that the presence of sexually receptive females increases the number of non-contact penile erections in male rats. This finding is consistent and stable and results in a high enough basal level of non-contact penile erections such that an impairment could result in statistical significance. Therefore, the inventors utilized these parameters to conduct studies evaluating antidepressant treatment on non-contact penile erections.

While penile erection is only one of many complex components of sexual functioning, we demonstrate that a lack of penile erections can accurately represent the gestault of sexual dysfunction.

Behavioral models which have been used in the past to evaluate antidepressant induced sexual dysfunction generally consist of paired mating studies and qualitative analyses of numerous behaviors (Waldinger et al., 2002; Matuszcyk et al., 1998; Mos et al., 1999; Frank et al., 2000). Others have reported the increased incidence of penile erections in male rats placed in the presence of an inaccessible female that can be seen, heard, and smelled, but not touched (Sachs et al., 1994; Melis et al., 1998), however, the methods of evaluating non-contact penile erections are not believed to have been applied in a context to evaluate sexual dysfunction associated with antidepressant treatment.

The doses in the studies described herein were doses which have been previously reported to demonstrate antidepressant-like effects in behavioral tests predictive of antidepressant activity in rats (Reneric et al., 1998; West et al., 1998) and have also been evaluated in our laboratory as doses which reverse the hyperactivity associated with olfactory bulbectomy in rats after chronic treatment (unpublished).

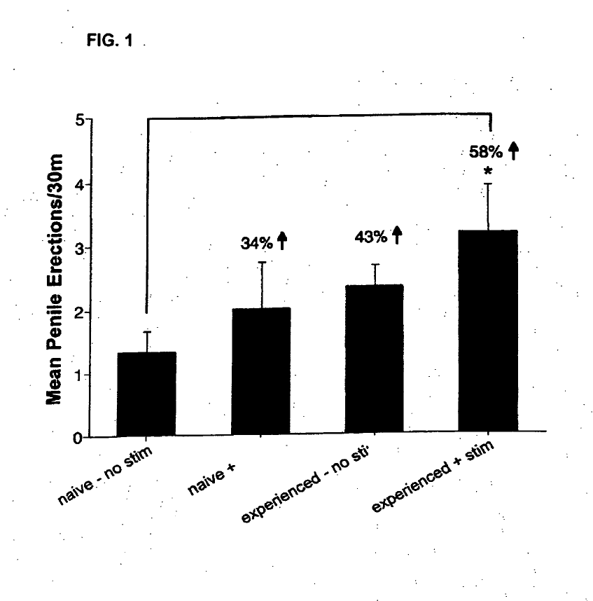

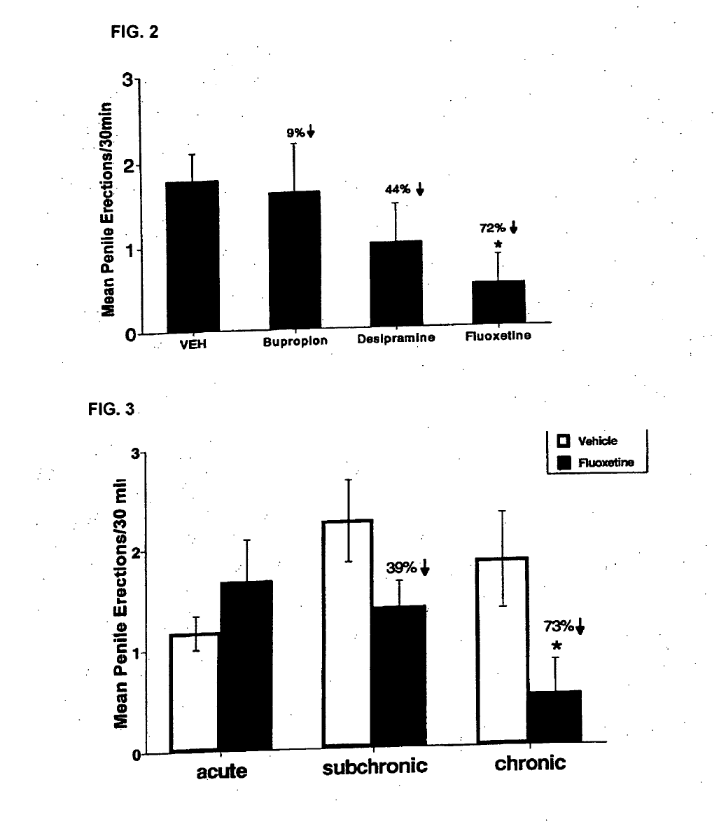

Data recorded from the studies demonstrated the following: (1) the number of penile erections is increased in sexually experienced males relative to naive males and in the presence of receptive females relative to no females present (see FIG. 1), (2) antidepressant treatment to rats results in a reduction of sexual dysfunction as measured by non-contact penile erections relative to vehicle treated subjects which patterns reports for antidepressant-induced sexual dysfunction in humans (see FIG. 2), and (3) the time course for the onset of antidepressant-induced sexual dysfunction in this behavioral model is consistent with a time course for the onset of antidepressant-induced sexual dysfunction as reported in humans (see FIGS. 3-5). Thus, it was unexpectedly determined that antidepressant-induced sexual dysfunction, as measured by a reduction in the number of penile erections, mirrors clinical reports for the rank order of sexual impairment attributed to the particular classes of antidepressants evaluated (SSRIs>tricyclic antidepressants(TCAs)>dopamine and norephinephrine reuptake inhibitor (D/NRI)).

Important to note, acute administration of bupropion produced hyperactivity in treated subjects relative to vehicle-treated subjects and likely masked the ability of these animals to demonstrate penile erections. This behavior may be due to increases of dopamine resulting from acute administration of this drug. Interestingly, this hyperactivity was not observed in animals treated for 7 and 14 days.

Methods of the Invention

The present invention includes methods of evaluating substances (or quantities of substances) for their effects on sexual function and methods of preventing/treating sexual dysfunction (or enhancing sexual function) using the substances or quantities of substances indicated by said methods for evaluating substances (e.g., as pharmaceutical compositions).

According to an embodiment, the present invention contemplates using an animal model to evaluate substances for their effect on sexual function in humans and other mammals. As used herein, the term “substances” is intended in its broadest sense and includes any compounds, combinations of compounds or compositions containing the compounds, such as pharmaceutical compositions, without limitation. Also contemplated by the present invention is a method of evaluating different quantities of substances for their effect on sexual function in humans and other mammals.

For instance, the methods of the invention may be used to evaluate substances for impairment and/or benefit to sexual function. For example, the methods of the present invention may be used to screen a library of compounds or compositions to identify compounds or compositions for use in improving sexual function or preventing and/or treating sexual dysfunction, including assisting in determining appropriate quantities of said substances for the objective. Likewise, the methods of the present invention may be used for screening compounds or compositions to assess their possible side effects prior to administration to subjects (e.g., patients, etc.). Further, the methods may be used to evaluate combinations of substances for the ability of a substance or group of substances (e.g., reversible agent(s)) to negate or neutralize the negative effects of another substance or group of substances on sexual function. Additionally, the methods may be used to evaluate the quantity or dosage of substances for the corresponding effect on sexual function.

According to an embodiment, the methods of the present invention comprise: (a) administering the composition to one or more male mammals, (b) placing the one or more male mammals in a testing arena comprising one or more female mammals, wherein the males are separated from the females by a separating means which prevents physical contact between the male and female mammals, wherein the separating means does not inhibit or prevent the exchange of olfactory, auditory or visual stimuli between the male and females, and (c) recording the number of observed penile erections in the one or more male mammals; wherein a change in the number of penile erections, relative to control male mammals receiving a placebo composition, indicates that the pharmaceutical composition has altered the sexual function of the one or more male mammals.

According to another embodiment of the present invention, the method comprises: (a) administering a first composition which induces sexual dysfunction to one or more male mammals, (b) placing the one or more male mammals of step (a) in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females, (c) recording the number of observed penile erections in the male mammals of step (b), wherein a decrease in the number of penile erections, relative to control males receiving a placebo composition, indicates that the first composition has induced a sexual dysfunction in the male mammal, (d) administering a second composition to the same male mammals used in step (a), (e) placing the one or more male mammals of step (d) in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females, and (f) recording the number of observed penile erections in the male mammals of step (e); wherein an increase in the number of penile erections, relative to recordings of step (c), indicates that the second composition reverses the sexual dysfunction induced by the first pharmaceutical composition.

Any animals suitable for testing as would be known to persons skilled in the art are contemplated for use in the present invention. Preferably, the animals are mammals. More preferably, the animals are non-human primates. Even more preferably, the animals are rodents. Even more preferably, the animals are rats. Preferably, the male rats and/or female rats are Sprague-Dawley, Wister or Long-Evans rats.

The animals may be of any age. Preferably, the male mammals are sexually mature males. More preferably, the males and females are sexually mature at the time of mating. Preferably, the females are sexually receptive, wherein sexually receptive is defined as a natural estrous cycle and or as a chemically induced estrous cycle. Even more preferably, the male animals (e.g., rats) are about 8 to 10 weeks of age.

Any suitable number of animals may be used. Preferably, about 1 to about 1,000 female mammals are present in the testing arena. More preferably, about 1 to about 100 female mammals are present in the testing arena. Even more preferably, at least about 5 to about 50 female mammals are present in the testing arena. Even more preferably, about 5 to about 20 female animals are present in the testing arena. Even more preferably, 5 to 10 female animals are present in the testing arena.

Prior to testing the male animals are preferably housed in rooms separate from the female animals at all times with the exception of overnight mating sessions. The housing cage is preferably a different in texture, shape, and/or size than the testing arena (described below).

The animals are preferably allowed a single overnight mating session with sexually receptive female rats. Preferably, male rats are separated from female rats at the conclusion of mating sessions and not exposed to the presence of any female rats until the test session (described below). Each of the one or more male mammals may mate with one or more female mammals prior to being administered the substance to be tested (or vehicle). The male mammals may further mate one or more times with one or more female mammals during administration of the substance.

Prior to the test session and immediately following the mating session, the substance (vehicle or substance to be tested, e.g., pharmaceutical or other compositions as described below) is administered during the required evaluation period. A non-limiting list of exemplary substances for testing (e.g., antidepressants, anxiolytics, antipsychotics, or combinations thereof, etc.) is provided below in the discussion of compositions of the invention. These exemplary substances are provided merely for example and the invention is not intended to be limited thereto. Any suitable substance for testing as would be understood by a person skilled in the art, based upon the guidance provided herein, is contemplated by the present invention.

Preferably, prior to the test session and immediately following the mating session, vehicle or substance to be tested is administered daily for the required evaluation period. Preferably, the evaluation period ranges from about 7 to about 14 days. More preferably, the evaluation period is 7 days for subchronic and 14 days for chronic.

Animals are preferably tested only on the final day of the treatment period (the period during which the substance being tested is administered to the animal). Testing sessions preferably occur during the light phase of the circadian period, but preferably do not begin earlier than 6 hours after lights on. Even more preferably, subjects began test sessions no earlier than noon.

The substance is preferably administered about 15 to 90 minutes, more preferably about 20 to 75 minutes and even more preferably about 30 or 60 minutes prior to the start of the test session. The test session will preferably last about 15 to 90 minutes, more preferably 20 to 75 minutes or even more preferably about 30 to 60 minutes in duration, with the number of non-contact penile erections quantified per animal.

The composition is administered over the course of a suitable period of time, as would be understood by a person skilled in the art based upon the guidance provided herein. Preferably, the composition is administered to the one or more male mammals daily for about 1 to about 30 days prior to placement of the one or more males in the testing arena. More preferably, the composition is administered to the one or more male mammals daily for about 5 to about 20 days prior to placement of the one or more males in the testing arena. Even more preferably, the pharmaceutical composition is administered to the one or more male mammals daily for about 7 to about 14 days prior to placement of the one or more males in the testing arena.

According to an embodiment the amount of the pharmaceutical composition is administered to the one or more male mammals daily for about 7 days. According to another embodiment, the amount of the pharmaceutical composition is administered to the one or more male mammals daily for about 14 days.

Any substance may be tested using the methods of the invention. Preferably, the substance is a pharmaceutical composition. Preferably, the pharmaceutical composition comprises a small molecule. According to an embodiment the small molecule is an antidepressant, an anxiolytic or an antipsychotic. Preferably, the antidepressant is a serotonin reuptake inhibitor, a dopamine/norepinephrine reuptake inhibitor, a tricyclic or a norepinephrine reuptake inhibitor. According to an embodiment, the small molecule is a compound used to treat erectile dysfunction. The compositions of the invention are described in further detail below.

The testing environment is preferably designed so that males are separated from the females by a separating means which prevents physical contact between the male and female mammals, wherein the separating means does not inhibit or prevent the exchange of olfactory, auditory or visual stimuli between the male and females. Thus, although the male animals cannot physically touch the females, they can hear, see and smell the female animals. The separating means is described in further detail below in the discussion of the apparatus of the invention.

The testing environment preferably comprises a room supplied with whitenoise as background so animals are not distracted by random or external noise. Preferably, the whitenoise is at about 65 dB. Receptive female rats (no fewer than 20) are dispersed throughout the room and additionally placed directly behind the testing arena. Receptive females provide additional stimuli of which the test subjects can see, smell, and hear, but cannot touch. Preferably, the testing arena comprises a solid Plexiglas enclosure with no bedding, measuring 10′/2″ wide×19″ deep×8″ high with a perforated top that increases the ceiling height to 11 inches. The testing arena preferably differs significantly from the homecage environment. In between subjects, the testing arena is preferably wiped clean of gross materials but is not sanitized in order to promote as much sexually related odors and stimuli as possible.

The animals are monitored during the study in any suitable manner as known to persons skilled in the art. Preferably, the animals are monitored to quantify the observed penile erections in the males. For instance, the animals are monitored for a decrease in the number of observed penile erections in the males receiving the pharmaceutical composition, relative to the males receiving placebo to determine whether the pharmaceutical composition induces sexual dysfunction in the male mammal. Likewise, the animals are monitored for an increase in the number of observed penile erections in the males receiving the pharmaceutical composition, relative to the males receiving placebo, indicates that the pharmaceutical composition enhances sexual function in the male mammal. Preferably, the quantity of penile erections is measured for about a 1 to 120 minute period of time. More preferably, the quantity of penile erections is measured for about a 1 to 90 minute period of time. Even more preferably, the quantity of penile erections is measured for about a 1 to 60 minute period of time. Even more preferably, the quantity of penile erections is measured for about a 1 to 30 minute period of time. Even more preferably, the quantity of penile erections is measured for about a 1 to 15 minute period of time.

The penile erections may be observed during the light and/or dark phase of the circadian period. Preferably, the quantity of penile erections is measured during light phase of the circadian period no earlier than 6 hours after lights on.

In addition to the methods for evaluating substances for their effects on sexual function, the present invention also provides methods for preventing and/or treating sexual dysfunction (or enhancing sexual function) by using the substances or quantities of substances as indicated by the above-described screening methods of the present invention (e.g., in pharmaceutical compositions, as described below).

Compositions of the Invention

The present invention contemplates compositions comprising substances or quantities of substances identified by methods of the present invention. For example, the compositions of the inventions comprise a substance or quantity of a substance for purposes of testing using the methods of the invention to evaluate substances as described above. Also, the present invention contemplates compositions (e.g., pharmaceutical compositions) or any combinations of compositions for treating and/or preventing sexual dysfunction and/or enhancing sexual function in a subject. Further, the present invention contemplates compositions or any combination of compositions for reversing or ameliorating the effects of other substances, for example, compositions that reverse the effects of antidepressant-induced sexual dysfunction, without limitation. For instance, the present invention contemplates compositions (or combinations of same) that reverse (or partially reverse) or in other ways ameliorate or mitigate the effects of another composition that a subject is administered (or otherwise exposed to) prior to, during and/or after administration of said first composition.

Any suitable substance, or combination of substances, is contemplated by the present invention for evaluation according to the methods of the invention. Any suitable substance, or combination of substances, is contemplated for inclusion in compositions of the invention, such as pharmaceutical compositions, for administration to subjects.

According to an embodiment, the compositions of the present invention comprise a small molecule. According to an embodiment, the small molecule is an antidepressant, an anxiolytic, an antipsychotic, or any combination thereof, without limitation. Any suitable such small molecules are contemplated by the present invention.

According to an embodiment of the present invention, the small molecule may be a compound used to treat erectile dysfunction, for example, without limitation. The present invention contemplates any reversible agent, that is, a agent that reverses the effects caused by another agent, such as impairment of sexual dysfunction. For example, any substance or combination of substances (and compositions comprising same) that reverse the effects of antidepressant induced sexual dysfunction.

Suitable antidepressants include, for example, seratonin reuptake inhibitors (SRIS), adrenergic reuptake inhibitors (NRIs), combined serotonin-adrenergic reuptake inhibitors (SNRIs), monoamine oxidase inhibitors (MAOs), reversible inhibitors of monoamine oxidase (RIMAs), phosphodiesterase-4 (PDE4) inhibitors, corticotropin releasing factor (CRF) antagonists, alpha.-adrenoreceptor antagonists or other compounds including atypical antidepressants. Additional antidepressants that may be useful in the practice of the present invention include super neurotransmitter uptake blockers (SNUBs; e.g., NS-2389 from GlaxoSmithKline and Neurosearch; (R)-DDMA from Sepracor), and/or substance P/neurokinin receptor antagonists (e.g., aprepitant/MK-869 from Merck; NKP-608 from Novartis; CPI-122721 from Pfizer; R673 from Roche; TAK637 from Takeda; and GW-97599 from GlaxoSmithKline).

Another class of antidepressant agents that may be used in the present invention are noradrenergic and specific serotonergic antidepressants (NaSSAs). A suitable example of a NaSSA is mirtazepine.

Suitable NRIs that may be used in the present invention include tertiary amine tricyclics and secondary amine tricyclics. Suitable examples of tertiary amine tricyclics include: amitriptyline, clomipramine, doxepin, duloxetine, imipramine (See U.S. Pat. No. 2,554,736, incorporated herein by reference in its entirety) and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Another NRI that may be used in the present invention is reboxetine (Edronax™; 2-[.alpha.-(2-ethoxy)phenoxy-benzyl]morpholine, usually administered as the racemate; See U.S. Pat. No. 4,229,449, incorporated herein by reference in its entirety).

Suitable SSRIs that may be used in the present invention include: citalopram (1-[3-(dimethylamino)propyl]-(4-fluorophenyl)-1,3-dihydr-o-5-isobenzofurancarbonitrile; See U.S. Pat. No. 4,136,193; Christensen et al., Eur. J. Pharmacol. 41:153, 1977; Dufour et al., Int. Clin. Psychopharmacol. 2:225, 1987; Timmerman et al., ibid., 239, each of which is incorporated herein by reference in its entirety); fluoxetine (N-methyl-3-(p-trifluoromethylphenoxy)-3-phenylpropylamine, marketed in the hydrochloride salt form and as the racemic mixture of its two isoforms; see, for example, U.S. Pat. No. 4,314,081; Robertson et al., J. Med. Chem. 31:1412, 1988, each of which is incorporated herein by reference); fluoxetine/olanzapine in combination; duloxetine (N-methyl-3-(1-naphthalenyloxy)-3-(2-thienyl)propanamine, usually administered as the hydrochloride salt and as the (+) enantiomer; see U.S. Pat. No. 4,956,388, incorporated herein by reference in its entirety); fluvoxamine (5-methoxy-1-[4-(trifluoromethyl)phenyl]-1-pentanone O-(2-aminoethyl)oxime; See U.S. Pat. No. 4,085,225; Claassen et al., Brit. J. Pharmacol. 60:505, 1977; De Wilde et al., J. Affective Disord. 4:249, 1982; Benfield et al., Drugs 32:313, 1986, each of which is incorporated herein by reference in its entirety); paroxetine (trans-(−)-3-[(1,3-benzodioxol-5-yloxy)methyl]-4-(4-fluo-rophenyl)piperidine; See U.S. Pat. No. 3,912,743; U.S. Pat. No. 4,007,196; Lassen, Eur. J. Pharmacol. 47:351, 1978; Hassan et al., Brit. J. Clin. Pharmacol. 19:705, 1985; Laursen et al., Acta Psychiat. Scand. 71:249, 1985; Battegay et al., Neuropsychobiology 13:31, 1985, each of which is incorporated herein by reference in its entirety); sertraline, (1S-cis)-4-(3,4-dichlorophenyl)-1,2,3,4-tetrahydro-N-methyl-1-naphthylamine hydrochloride; See U.S. Pat. No. 4,536,518, incorporated herein by reference in its entirety); escitalopram (see U.S. Pat. No. RE34,712); new 5HT1A agonists variza, alnespirone, gepirone, sunepitron, MKC242, vilazodone, eptapirone, and ORG12962 from Organon; new 5HT1A antagonists such as robalzotan; new 5-HT1B agonists such as elzasonan; new 5HT2 antagonists such as YM-992 (from Yamanouchi Pharmaceuticals) and nemifitide, and pharmaceutically acceptable salts thereof.

Suitable MAOIs that may be used in the present invention include: isocarboxazid, phenelzine, selegiline and tranylcypromine, and pharmaceutically acceptable salts thereof.

Suitable reversible MAOIs that may be used in the present invention include: moclobemide (4-chloro-N-[2-(4-morpholinyl)-ethyl]benzamide; See U.S. Pat. No. 4,210,754, incorporated herein by reference in its entirety), selegiline, and pharmaceutically acceptable salts thereof.

Suitable SNRIs that may be used in the present invention include venlafaxine (see U.S. Pat. No. 4,535,186, incorporated herein by reference in its entirety; see also U.S. Pat. Nos. 5,916,923, 6,274,171, 6,403,120, 6,419,958, 6,444,708, each of which is incorporated herein by reference in its entirety), and pharmaceutically acceptable salts and analogs, including the O-desmethylvenlafaxine succinate salt; milnacipran (N,N-diethyl-2-aminomethyl-1-phenylcyclopropariecarboxamide; see U.S. Pat. No. 4,478,836; Moret et al., Neuropharmacology 24:1211-19, 1985, each of which is incorporated herein by reference in its entirety); mirtazapine (see, for example, U.S. Pat. No. 5,178,878, the entire contents of which are incorporated herein by reference); nefazodone (available from Bristol Myers Squibb and Dr. Reddy Labs Inc.); duloxetine; and pharmaceutically acceptable salts thereof.

Suitable CRF antagonists that may be used in the present invention include those compounds described in International Patent Specification Nos. WO 94/13643, WO 94/13644, WO 94/13661, WO 94/13676 and WO 94/13677.

Suitable atypical antidepressants that may be used in the present invention include: buproprion (Welibutrin™; (.+−.)-1-(3-chlorophenyl)-2-[(1,1-dimethylethyl)amino]-1-propanone), lithium, nefazodone, trazodone and viloxazine, and pharmaceutically acceptable salts thereof. Another suitable atypical antidepressant is sibutramine.

Particular antidepressants that may be used in the present invention include, but are not limited to, adinazolam, alaproclate, alnespirone, amineptine, amitriptyline, amitriptyline/chlordiazepoxide combination, amoxapine, aprepitant, atipamezole, azamianserin, bazinaprine, befuraline, bifemelane, binodaline, bipenamol, brofaromine, buproprion, caroxazone, cericlamine, cianopramine, cimoxatone, citalopram, clemeprol, clomipramine, clovoxamine, dazepinil, deanol, demexiptiline, desipramine, O-desmethylvenlafaxine, dibenzepin, dothiepin, doxepin, droxidopa, duloxetine, elzasonan, enefexine, eptapirone, escitalopram, estazolam, etoperidone, femoxetine, fengabine, fezolamine, fluotracen, fluoxetine, fluvoxamine, gepirone, idazoxan, imipramine, indalpine, indeloxazine, iprindole, isocarboxazid, levoprotiline, litoxetine, lofepramine, maprotiline, medifoxamine, metapramine, metralindole, mianserin, milnacipran, minaprine, mirtazapine, moclobemide, montirelin, nebracetam, nefopam, nefozodine, nemititide, nialamide, nomifensine, norfluoxetine, nortriptyline, orotirelin, oxaflozane, paroxetine, pheneizine, pinazepam, pirlindone, pizotyline, protryptiline, reboxetine, ritanserin, robalzotan, rolipram, selegiline, sercloremine, sertraline, setiptiline, sibutramine, sulbutiamine, sulpiride, sunepitron, teniloxazine, thozalinone, thymoliberin, tianeptine, tiflucarbine, tofenacin, tofisopam, toloxatone, tomoxetine, tranylcypromine, trazodone, trimiprimine, venlafaxine, veralipride, vilazodone, viloxazine, viqualine, zimelidine and zometrapine, and pharmaceutically acceptable salts thereof, and St. John's wort herb, or Hypencuin perforatum, extracts thereof, or any combinations thereof.

Preferably, the antidepressant is any of seratonin reuptake inhibitors (SRIs), adrenergic reuptake inhibitors (NRIs), combined serotonin-adrenergic reuptake inhibitors (SNRIs), monoamine oxidase inhibitors (MAOIs), reversible inhibitors of monoamine oxidase (RIMAs), phosphodiesterase-4 (PDE4) inhibitors, corticotropin releasing factor (CRF) antagonists, alpha.-adrenoreceptor antagonists or other compounds including atypical antidepressants, super neurotransmitter uptake blockers and/or substance P/neurokinin receptor antagonists or any combination thereof. More preferaably, the antidepressant is a selective serotonin reuptake inhibitor (SSRI), a dopamine/norepinephrine reuptake inhibitor, a tricyclic, a norepinephrine reuptake inhibitor or any combination thereof.

Non-limiting exemplary anxiolytics include, without limitation, diazepam, alprazolam or a combination thereof.

Non-limiting exemplary antipsychotics include, without limitation, amisulpride, chlorpromazine, clozapine, haloperidol, olanzapine, quetiapine, risperidone, sulpiride, zotepine or any combination thereof.

The formulation of pharmaceutical compositions is well known to persons skilled in this field. Pharmaceutical compositions of the invention preferably include a pharmaceutically acceptable carrier. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibody. The preparation and use of pharmaceutically acceptable carriers is well known in the art. Except insofar as any conventional media or agent is incompatible with the active ingredient, use thereof in the immunogenic compositions of the present invention is contemplated.

Such compositions can be administered parenterally, e.g., by injection, either subcutaneously or intramuscularly, as well as orally or intranasally. Methods for intramuscular immunization are described by Wolff et al. and by Sedegah et al. Other modes of administration employ oral formulations, pulmonary formulations, suppositories, and transdermal applications, for example, without limitation. Oral formulations, for example, include such normally employed excipients as, for example, pharmaceutical grades of mannitol, lactose, starch, magnesium stearate, sodium saccharine, cellulose, magnesium carbonate, and the like, without limitation.

Where appropriate, the pharmaceutical compositions can be administered by inhalation, in the form of a suppository, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner. It is also possible to administer the agents of the present invention in sustained release formulations.

In some applications, generally in humans, oral administration of the compositions of the present invention is the preferred route, being the most convenient and in some cases able to avoid disadvantages associated with other routes of administration—such as those associated with intracavernosal (i.c.) administration. In circumstances where the recipient suffers from a swallowing disorder or from impairment of drug absorption after oral administration, the drug may be administered parenterally.

For veterinary use, the agent of the present invention is typically administered as a suitably acceptable formulation in accordance with normal veterinary practice and the veterinary surgeon will determine the dosing regimen and route of administration which will be most appropriate for a particular animal. However, as with human treatments, it may be possible to administer the agent alone for veterinary treatments.

Typically, the pharmaceutical compositions—which may be for human or animal usage—will comprise any one or more of a pharmaceutically acceptable diluent, carrier or excipient. The choice of pharmaceutical carrier, excipient or diluent can be selected with regard to the intended route of administration and standard Pharmaceutical practice. As indicated above, the pharmaceutical compositions may comprise as—or in addition to—the carrier, excipient or diluent any suitable binder(s), lubricant(s), suspending agent(s), coating agent(s) or solubilising agent(s).

Thus the invention provides a pharmaceutical composition comprising an agent of the present invention (or even a pharmaceutically acceptable salt thereof, or a pharmaceutically acceptable solvate thereof) together with a pharmaceutically acceptable diluent, excipient or carrier.

The present invention yet further relates to pharmaceutical compositions which may comprise a substance of the present invention alone or in combination with at least one other agent, such as stabilizing compound, and may be administered in any sterile, biocompatible pharmaceutical carrier, including, but not limited to, saline, buffered saline, dextrose, and water.

The pharmaceutical compositions of the present invention may be used in combination with one or more other pharmaceutically active agents. If it is administered by injection, the injection may be intravenous, subcutaneous, intramuscular, intraperitoneal or other means known in the art. The compositions of this invention may be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which the compositions of invention are introduced across an epidermal layer of cells may be employed. Administration means may include administration through mucous membranes, buccal administration, oral administration, dermal administration, inhalation administration, nasal administration and the like.

As is contemplated by the present invention, a physician will typically determine the actual dosage which will be most suitable for an individual patient and it will vary with the age, weight, sex and response of the particular patient.

Apparatus of the Invention

The present invention provides a testing arena suitable for carrying out the methods of the present invention. For instance, the apparatus of the present invention prevents physical contact between the male and female mammals, without inhibiting or preventing the exchange of olfactory, auditory and/or visual stimuli between the male and females.

According to an embodiment, the apparatus of the invention comprises: a separating means which prevents physical contact between the male and female mammals, but does not inhibit or prevent the exchange of olfactory, auditory or visual stimuli between the male and females.

The separating means is any barrier that prevents physical contact between the male and female mammals, but does not inhibit or prevent the exchange of olfactory, auditory or visual stimuli between the male and females. Preferably, the separating means is transparent on the sides and has perforations or an open space on top to permit the free flow of air and sound into and out of a solid enclosure, said solid enclosure having a sealable opening through which small animals may be placed within said solid enclosure. For example, a suitable separating means for the present invention is provided by Allentown Cage Equipment Company, Inc. of Allentown, N.J., Rat cage model # PC10147HT and cage top model # MBT1019HT

The separating means may be made of any material or combinations of materials. Preferably, the material is easy to clean and sanitize. Preferably, the separating means is a rigid structure such as glass. More preferably, the separating means is Plexiglas.

According to an embodiment, the separating mean is a first containing means containing a male mammal and a second containing means containing one or more female mammals. According to another embodiment, the separating means is a first containing means containing two or more male mammals and a second containing means containing the one or more female mammals. Preferably, the containing means is a cage.

According to an embodiment, the separating means is a dividing barrier placed into the testing arena, wherein a male mammal and the one or more female mammals are on opposite sides of the barrier. According to another embodiment, the separating means is a dividing barrier placed into the testing arena, wherein two or more male mammals and the one or more female mammals are on opposite sides of the barrier.

The separating means may take any suitable shape or size to accomplish the objectives of the method. Preferably, the separating means has a depth of about twice its width and a ceiling less than its width. More preferably, the separating means is about 10 inches to about 30 inches deep. Even more preferably, the separating means is about 19 inches deep. Even more preferably, the separating means is about 6 inches to about 15 inches wide. Even more preferably, the separating means is about 10½ inches wide. Preferably, the separating means has a ceiling of about 4 inches to about 14 inches. More preferably, the separating means has a ceiling of about 8 inches. Preferably, the internal dimensions of the separating means are about 10½ inches×19 inches×8 inches. More preferably, the external dimensions of the solid enclosure are about 10½ inches wide×19 inches deep×11 inches high.

The separating means is placed in any suitable environment for testing as would be understood by a person of skill in the art, based upon the guidance provided herein. Preferably, the testing environment is as described above in the discussion of the methods of the invention.

The following examples are included to illustrate the present invention and demonstrate preferred embodiments of the invention. It should be appreciated by those skilled in the art that the techniques disclosed in the examples which follow represent techniques discovered by the inventors to function well in the practice of the invention, and thus can be considered to constitute preferred modes for its practice. However, those of skill in the art should, in view of the present disclosure, appreciate that many changes can be made in the specific embodiments which are disclosed and still obtain a like or similar result without departing from the spirit and scope of the invention. the embodiments described herein are not limited to the specific conditions or details described in these examples. Thus, it should be understood that any and all references to a publicly available document, including a U.S. patent, are specifically incorporated by reference in their entirety.

### EXAMPLES

The doses of the antidepressants evaluated in the present studies were selected based on the reported efficacious dose ranges of these drugs in preclinical models predicting antidepressant-like activity in rats including olfacoty bulbectomy studies and forced swim test experiments (Reneric et al., 1998; West et al., 1998; Joly and Sanger, 1986). Fluoxetine HCl (Tocris), desipramine (Sigma; St. Louis, Mo.) and bupropion HCl (Toronto Research Chemicals, Canada) were dissolved in 0.9% sterile saline vehicle and injected intraperitoneally in a volume of 1 ml/kg once daily. On the day of behavioral testing, antidepressants were administered intraperitoneally (i.p.) as one hour pretreatments. For reversal experiments, apomorphine (Sigma; St. Louis, Mo.) was dissolved in 0.9% sterile saline vehicle and injected subcutaneously in a volume of 1 ml/kg. On the day of behavioral testing, both fluoxetine and apomorphine were co-administered as 0 minute pretreatments. 17B-estradiol benzoate and progesterone were purchased from Sigma (St. Louis, Mo.).

Data are presented as group means±sem. For statistical analysis, data was subjected to one way analysis of variance (ANOVA) with a least significant difference (LSD) post-hoc test as appropriate. Statistical significance was achieved when p<0.05.

### Example 1

### Effect of Sexual Experience or Inexperience in the Absence or Presence of Female Stimuli on Non-Contact Penile Erections in Male Rats

In order to establish the experimental parameters in which sexual dysfunction can be examined, non-contact penile erections were evaluated in sexually naive and sexually experienced rats in the absence and presence of female stimuli (n=5 per treatment group).

Intact male Sprague-Dawley rats (Charles River Laboratories) arrived at 7 weeks of age and were allowed to habituate for one week prior to sexual experience or behavioral testing. Rats were group housed in same-sex colony rooms with food and water provided ad libitum. All animals were maintained under a 12:12 light-dark cycle (lights on at 6:00 am) with behavioral experiments performed during the light hours beginning at noon.

Ovariectomized female Long-Evans rats (Charles River Laboratories) were individually housed and brought into behavioral estrus weekly by subcutaneous injection of 25 ug 17-B estradiol benzoate in 0.1 ml corn oil 48 hours prior to testing, followed by subcutaneous administration of 2.5 mg progesterone in 0.1 ml corn oil 4-6 hours before testing.

All studies were approved by the Institutional Animal Care and Use Committee at Wyeth and were in accordance with NIH guidelines for the care and use of animals.

For subjects assigned into the sexually experienced group, the following procedures were performed. Individual male rats were placed into a cage with a sexually receptive female for overnight mating sessions. The mating chamber which was the housing chamber of the female mating partner, was a Plexiglas cage measuring 10×12 inches, with food and water ad libitum. Mating pairs were randomly assigned. The interaction between the male and female rat was observed for the first hour. Only males that were observed for intromission within the first hour of paired mating sessions were included in behavioral studies. Following overnight mating sessions, males and females were separated from each other and placed back into same-sex colony rooms where they were group-housed until the time of behavioral testing.

To determine the effects of female stimuli on non-contact penile erections, sexually receptive females were brought into the testing room one hour prior to the start of behavioral testing in order to provide female visual, olfactory, and auditory stimuli for males. The testing arena for observing non-contact penile erections in male rats consisted of an empty rat size Plexiglas cage (11×10 inches) containing no bedding, with an aerated plastic lid and a total ceiling height of 12 inches. Males were observed for non-contact penile erections in individual test cages. A penile erection consisted of observation of the male rat in a hunched position grasping the penis with the forepaws and followed by a series of pelvic thrusts. The number of penile erections were quantified over a 30 minute observation session.

Subjects assigned to the nonstimuli group were observed in the same testing arena as described above, however there were no female rats present in the testing room.

Results:

Sexually naive rats (n=6) evaluated for non-contact penile erections in the absence of female stimuli exhibited a mean of 1.33+0.33 non-contact penile erections (FIG. 1). Addition of the presence of female stimuli to the testing area of naive rats resulted in a mean of 2.00+0.73 non-contact penile erections (n=5) which was a 34% increase in the number of penile erections relative to sexually naive rats in the absence of female stimuli (not significant (n.s.)). Sexually experienced rats in the absence of female stimuli (n=6) resulted in a mean of 2.33+0.33 non-contact penile erections which was an increase of 43%, relative to sexually naive subjects under the same conditions (n.s.). Sexually experienced males in the presence of female stimuli (n=6) exhibited a mean of 3.17+0.75 non-contact penile erections, which was a significant 58% increase in the number of non-contact penile erections relative to sexually naive rats with no female stimuli present (P<0.05).

FIG. 1 illustrates the effect of sexual experience versus sexual inexperience in the presence or absence of female stimuli on non-contact penile erections in male Sprague-Dawley rats. Naive−no stim=sexually naive male rats with no female stimuli present. Naive+stim=sexually naive male rats in the presence of sexually receptive female rats present. Experienced−no stim=sexually experienced male rats with no female stimuli present. Experienced+stim=sexually experienced male rats with sexually receptive females present. Data are expressed as means±sem. Percentage increases were calculated relative to naive−no stim group.

### Example 2

### Effects of Antidepressant Treatment on Non-Contact Penile Erections in Sexually Experienced Male Rats

Following sexual experience (as described above), male subjects (n=6-8) were administered either 10 mg/kg/day fluoxetine (FLX), 10 mg/kg/day desipramine (DMI), 20 mg/kg/day bupropion (BUP) or saline vehicle (VEH) for 1 day (acute), 7 days (subchronic), or 14 days (chronic). Non-contact penile erections were evaluated in 30 min test sessions in the presence of sexually receptive female rats as described in Example 1.

Results:

The mean number of non-contact penile erections produced by subjects chronically treated for 14 days with saline vehicle (n=14) was 1.79+0.32. Chronic (14 day) administration of bupropion (20 mg/kg/day, i.p.; n=8) resulted in a mean of 1.63+0.57 non-contact penile erections which was a 9% decrease in the number of non-contact penile erections relative to vehicle-treated controls (n.s.). Chronic (14 day) desipramine treatment (10 mg/kg/day, i.p.; n=8) resulted in a mean of 1.00+0.46 non-contact penile erections which was a 44% reduction in the number of non-contact penile relative to vehicle-treated controls (n.s.). Chronic (14-day) fluoxetine treatment (10 mg/kg/day, i.p.; n=6) produced only 0.50+0.34 non-contact penile erections which was a significant 72% impairment (P<0.05) in the number of non-contact penile erections relative to vehicle-treated controls.

FIG. 2 illustrates the effects of vehicle, fluoxetine (10 mg/kg/day i.p.), desipramine (10 mg/kg/day, i.p.), and bupropion (20 mg/kg/day, i.p.) treatment after 14-day treatment on the mean number of non-contact penile erections of sexually-experienced male rats in a 30-min test session in the presence of a receptive female. Data are shown as group means±sem. *A statistically significant difference and/or % decrease relative to vehicle treatment.

Time Course of Fluoxetine Effects on Sexual Dysfunction:

There was no difference between the mean number of non-contact penile erections in vehicle treated groups regardless of time course. Acute administration of saline vehicle (n=6) produced 1.17+0.17 non-contact penile erections. Acute fluoxetine treatment (10 mg/kg, i.p.; n=6) resulted in a mean of 1.67+0.42 non-contact penile erections which was a 29% increase in the number of non-contact penile erections relative to vehicle-treated subjects (n.s.). Subchronic (7 day) vehicle treatment (n=8) produced 2.25+0.41 non-contact penile erections. Subchronic (7 day) fluoxetine treatment (10 mg/kg/day, i.p.; n=8) resulted in a mean of 1.38+0.26 non-contact penile erections which was a 39% reduction in the number of non-contact penile erections relative to vehicle-treated controls (n.s.). Chronic (14 day) vehicle treatment (n=6) produced 1.83+0.48 non-contact penile erections. Chronic (14-day) fluoxetine treatment (10 mg/kg/day, i.p.; n=6) resulted in a mean of 0.50+0.34 non-contact penile erections which was a significant 73% decrease relative to vehicle-treated animals in the number of non-contact penile erections (P<0.05, FIG. 3).

FIG. 3 illustrates the effects of vehicle or fluoxetine (10 mg/kg/day, i.p.) following acute, subchronic, and chronic treatment on the number of non-contact penile erections in sexually-experienced males in 30-min test sessions in the presence of receptive females. Data are expressed as means±sem. *A statistically significant difference compared with the corresponding vehicle treatment.

Time Course for Desipramine Effects on Sexual Dysfunction:

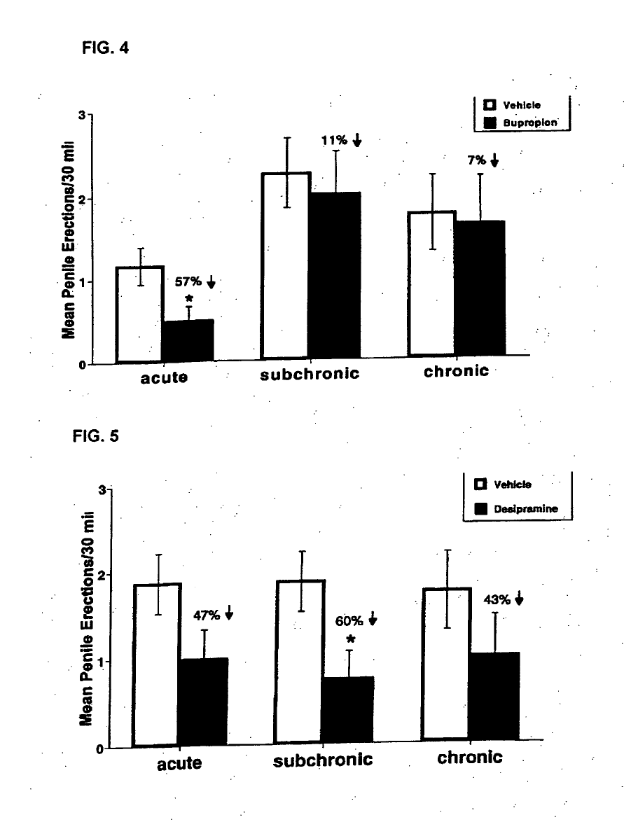

There was no difference between the mean number of non-contact penile erections in vehicle treated groups regardless of time course. Acute administration of saline vehicle (n=8) produced 1.88+0.35 non-contact penile erections. Acute desipramine treatment (10 mg/kg, i.p.; n=8) resulted in a mean of 1.00+0.33 non-contact penile erections which was a 47% decrease in the number of non-contact penile erections relative to vehicle-treated subjects (n.s.). Subchronic (7 day) vehicle treatment (n=8) produced 1.88+0.35 non-contact penile erections. Subchronic (7 day) desipramine treatment (10 mg/kg/day, i.p.; n=8) resulted in a mean of 0.75+0.31 non-contact penile erections which was a significant 60% reduction in the number of non-contact penile erections relative to vehicle-treated controls (P<0.05). Chronic (14 day) vehicle treatment (n=8) produced 1.75+0.45 non-contact penile erections. Chronic (14-day) desipramine treatment (10 mg/kg/day, i.p.; n=8) resulted in a mean of 1.00+0.46 non-contact penile erections which was an insignificant 43% decrease relative to vehicle-treated animals in the number of non-contact penile erections (n.s., FIG. 4).

FIG. 4 illustrates the effects of vehicle or desipramine (10 mg/kg/day, i.p.) following acute, subchronic, and chronic treatment on the number of non-contact penile erections in sexually-experienced males in 30 min test sessions in the presence of receptive females. Data are expressed as means±sem. *A statistically significant difference compared with the corresponding vehicle treatment.

Time Course of Bupropion Treatment on Sexual Dysfunction:

There was no difference between the mean number of non-contact penile erections in vehicle treated groups regardless of time course. Acute administration of saline vehicle (n=8) produced 1.17+0.17 non-contact penile erections. Acute bupropion treatment (20 mg/kg, i.p.; n=8) resulted in a mean of 0.50+0.22 non-contact penile erections which was a significant 57% decrease in the number of non-contact penile erections relative to vehicle-treated subjects (P<0.05) Subjects treated with acute bupropion were observed with increased locomotor activity relative to vehicle-treated controls. Subchronic (7 day) vehicle treatment (n=8) produced 2.25+0.41 non-contact penile erections. Subchronic (7 day) bupropion treatment (20 mg/kg/day, i.p.; n=8) resulted in a mean of 2.00+0.50 non-contact penile erections which was only an 11% reduction in the number of non-contact penile erections relative to vehicle-treated controls (n.s.). Chronic (14 day) vehicle treatment (n=8) produced 1.75+0.45 non-contact penile erections. Chronic (14-day) bupropion treatment (20 mg/kg/day, i.p.; n=8) resulted in a mean of 1.63+0.57 non-contact penile erections which was only a minimal 7% decrease relative to vehicle-treated animals in the number of non-contact penile erections (n.s., FIG. 5). Interestingly, the increased locomotor activity observed following acute bupropion treatment was not observed in the subjects treated subchronically and chronically.

FIG. 5 illustrates the effects of vehicle or bupropion (20 mg/kg/day, i.p.) following acute, subchronic, and chronic treatment on the number of non-contact penile erections in sexually-experienced males in 30 min test sessions in the presence of receptive females. Data are expressed as means±sem. *A statistically significant difference compared with the corresponding vehicle treatment.

### Example 3

### Ability of the Dopamine Agonist Apomorphine to Reverse the Deficits on Non-Contact Penile Erections in Sexually Experienced Male Rats Produced by Antidepressant Treatment

Following sexual experience (as described above), male subjects (n=8) were administered either 10 mg/kg/day fluoxetine (FLX) or saline vehicle (VEH) for 14 days. On the 14th day, fluoxetine treatment was co-administered with either saline or 0.1 mg/kg or 0.3 mg/kg apomorphine (sc). Non-contact penile erections were evaluated in 30 min test sessions in the presence of sexually receptive female rats as described in experiment 1.

Results:

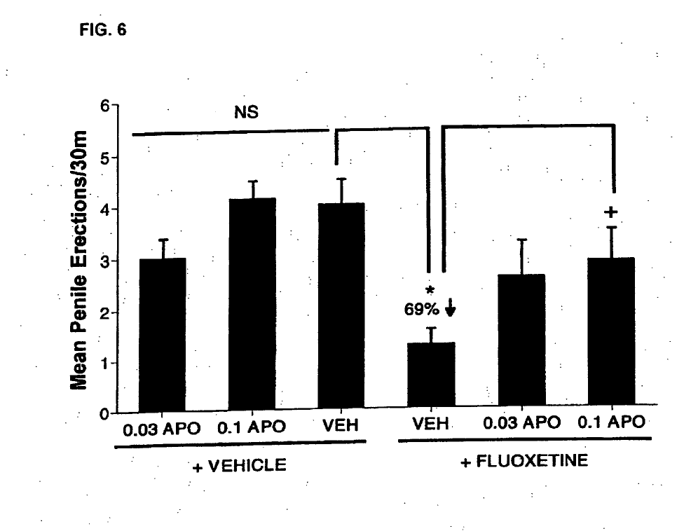

The reversal of the fluoxetine-induced deficits on non-contact penile erections in sexually experienced male rats by acute apomorphine treatment was demonstrated. The mean number of non-contact penile erections produced by subjects chronically treated for 14 days with saline vehicle (i.p.) co-administered with acute saline vehicle (subcutaneous (s.c.); n=8) was 4.00+0.49. Chronic (14 day) administration of fluoxetine (10 mg/kg/day, i.p.) co-administered with acute saline vehicle (s.c.; n=8) resulted in a mean of 1.25+0.31 non-contact penile erections which was a significant 69% decrease in the number of non-contact penile erections relative to vehicle-treated controls (P<0.001). Chronic (14 day) administration of fluoxetine (10 mg/kg/day, i.p.) co-administered with acute treatment of 0.03 mg/kg apomorphine (s.c.; n=8) resulted in a mean of 2.57+0.69 non-contact penile erections which was a 51.4% reversal of the number of non-contact penile erections relative to subjects in the acute vehicle+chronic fluoxetine treated group (P=0.0622). Chronic (14 day) administration of fluoxetine (10 mg/kg/day, i.p.) co-administered with acute treatment of 0.1 mg/kg apomorphine (s.c.; n=8) resulted in a mean of 2.88+0.61 non-contact penile erections which was a significant 56.5% reversal of the number of non-contact penile erections relative to subjects in the acute vehicle+chronic fluoxetine treated group (P<0.05). Acute administration of 0.03 mg/kg or 0.1 mg/kg apomorphine (s.c.) co-administered with chronic (14 day) saline vehicle treatment (i.p.) resulted in a mean 3.00+0.38 and 4.13+0.35 respectively, number of non-contact penile erections which was not different from acute vehicle+chronic vehicle treated subjects (FIG. 6).

FIG. 6 illustrates the effects of acute apomorphine treatment (0, 0.03, or 0.1 mg/kg, s.c.) co-administered with chronic (14 day) fluoxetine (10 mg/kg/day, i.p.) on the number of non-contact penile erections in sexually-experienced males in 30 min test sessions in the presence of receptive females. Data are expressed as means±sem. *A statistically significant difference compared with the corresponding vehicle treatment. +A statistically significant difference compared with the corresponding fluoxetine treatment.

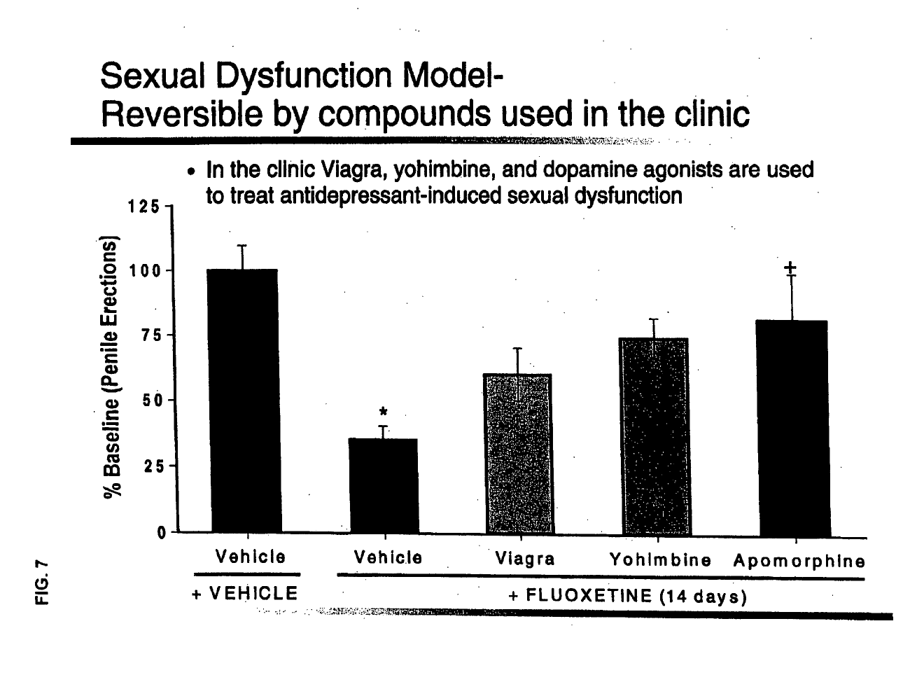

In a similar study, the ability of additional substances to reverse (or at least partially reverse) the effects of antidepressant-induced sexual dysfunction was evaluated. Sildenafil citrate (available as VIAGRA from Pfizer Inc. of New York, N.Y.), yohimbine and dopamine agonists were studied in the foregoing manner. The results of the study are provided in FIG. 7 which illustrates the effects of treatment with the studied compositions co-administered with fluoxetine (over 14 days) on the number of non-contact penile erections in sexually-experienced males. As shown in FIG. 7, the ability of the tested pharmaceutical compositions to reverse to effects of the fluoxetine is demonstrated.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A method for evaluating the effect of a composition on the sexual function of a male mammal, the method comprising:
(a) administering a composition to one or more male mammals,
(b) placing the one or more male mammals in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females, and
(c) recording the number of observed penile erections in the one or more male mammals;
wherein a change in the number of penile erections, relative to control male mammals receiving a placebo composition, indicates that the pharmaceutical composition has altered the sexual function of the one or more male mammals.

2. The method of claim 1, wherein a decrease in the number of observed penile erections in the males receiving the composition, relative to the males receiving placebo, indicates that the pharmaceutical composition induces sexual dysfunction in the male mammal.

3. The method of claim 1, wherein an increase in the number of observed penile erections in the males receiving the composition, relative to the males receiving placebo, indicates that the composition enhances sexual function in the male mammal.

4. The method of claim 1, wherein the composition comprises a small molecule.

5. The method of claim 4, wherein the small molecule is an antidepressant, an anxiolytic, an antipsychotic or any combination thereof.

6. The method of claim 5, wherein the antidepressant is a serotonin reuptake inhibitor, a dopamine/norepinephrine reuptake inhibitor, a tricyclic, a norepinephrine reuptake inhibitor or any combination thereof.

7. The method of claim 4, wherein the small molecule is a compound used to treat erectile dysfunction.

8. The method of claim 1, wherein the composition is administered to the one or more male mammals daily for about 1 to about 30 days prior to placement of the one or more males in the testing arena.

9. The method of claim 1, wherein the composition is administered to the one or more male mammals daily for about 5 to about 20 days prior to placement of the one or more males in the testing arena.

10. The method of claim 1, wherein the composition is administered to the one or more male mammals daily for about 7 to about 14 days prior to placement of the one or more males in the testing arena.

11. The method of claim 1, wherein the amount of the composition is administered to the one or more male mammals daily for about 7 days.

12. The method of claim 1, wherein the amount of the composition is administered to the one or more male mammals daily for about 14 days.

13. The method of claim 1, wherein the separating means is a first containing means containing a male mammal and a second cage containing means containing the one or more female mammals.

14. The method according to claim 13, wherein the containing means is a cage.

15. The method of claim 1, wherein the separating means is a first containing means containing two or more male mammals and a second containing means containing the one or more female mammals.

16. The method according to claim 15, wherein the containing means is a cage.

17. The method of claim 1, wherein the separating means is a dividing barrier placed into the testing arena, wherein a male mammal and the one or more female mammals are on opposite sides of the barrier.

18. The method according to claim 17, wherein the dividing barrier is glass or plexi-glass.

19. The method of claim 1, wherein the separating means is a dividing barrier placed into the testing arena, wherein two or more male mammals and the one or more female mammals are on opposite sides of the barrier.

20. The method according to claim 19, wherein the dividing barrier is glass or plexi-glass.

21. The method of claim 1, wherein the male and female mammals are rodents.

22. The method of claim 21, wherein the rodents are rats.

23. The method of claim 22, wherein the male rats are Sprague-Dawley rats, Wister rats, Long-Evans rats or a combination thereof.

24. The method of claim 22, wherein the female rats are Sprague-Dawley rats, Wister rats, Long-Evans rats or a combination thereof.

25. The method of claim 22, wherein the rats are about 8 to 10 weeks of age.

26. The method of claim 1, wherein the male and female mammals are non-human primates.

27. The method of claim 1, wherein 5 to 10 female mammals are present in the testing arena.

28. The method of claim 1, wherein 5 to 20 female mammals are present in the testing arena.

29. The method of claim 1, wherein 1 to 100 female mammals are present in the testing arena.

30. The method of claim 1, wherein 1 to 1,000 female mammals are present in the testing arena.

31. The method of claim 1, wherein the male mammals are further defined as sexually mature males.

32. The method of claim 22, wherein the male mammals are about 8 to 10 weeks of age.

33. The method of claim 1, wherein the quantity of penile erections is measured for about a 1 to 120 minute period of time.

34. The method of claim 33, wherein the quantity of penile erections is measured for about a 1 to 90 minute period of time.

35. The method of claim 33, wherein the quantity of penile erections is measured for about a 1 to 60 minute period of time.

36. The method of claim 33, wherein the quantity of penile erections is measured for about a 1 to 30 minute period of time.

37. The method of claim 33, wherein the quantity of penile erections is measured for about a 1 to 15 minute period of time.

38. The method of claim 1, wherein the quantity of penile erections is measured during light phase of the circadian period.

39. The method of claim 38, wherein the quantity of penile erections is measured during light phase of the circadian period no earlier than 6 hours after lights on.

40. The method of claim 1, wherein the quantity of penile erections is measured during dark phase of the circadian period.

41. The method of claim 1, wherein each of the one or more male mammals are mated with one or more female mammals prior to being administered the pharmaceutical composition.

42. The method of claim 41, wherein the male mammals further mate one or more times with one or more female mammals during administration of the pharmaceutical composition.

43. The method of claim 1, wherein the testing arena is supplied with a white noise background.

44. The method of claim 43, wherein the white noise background is at about 65 dB.

45. The method of claim 1, wherein the males and females are sexually mature at the time of mating.

46. The method of claim 1, wherein the females are sexually receptive, wherein sexually receptive is defined as a natural estrous cycle.

47. The method of claim 1, wherein the females are sexually receptive, wherein sexually receptive is defined as a chemically induced estrous cycle.

48. A method for evaluating the effect of a composition on the alleviation or reversal of a sexual dysfunction in a male mammal, the method comprising the steps of:
(a) administering a first composition which induces sexual dysfunction to one or more male mammals,
(b) placing the one or more male mammals of step (a) in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females,
(c) recording the number of observed penile erections in the male mammals of step (b), wherein a decrease in the number of penile erections, relative to control males receiving a placebo composition, indicates that the first composition has induced a sexual dysfunction in the male mammal,
(d) administering a second composition to the same male mammals used in step (a),
(e) placing the one or more male mammals of step (d) in a testing arena comprising one or more female mammals, wherein the males are separated from the female by a separating means which prevents physical contact between the male and female mammals, wherein the separating means does not inhibit or prevent the exchange of olfactory, auditory or visual stimuli between the male and females, and
(f) recording the number of observed penile erections in the male mammals of step (e);
wherein an increase in the number of penile erections, relative to recordings of step (c), indicates that the second composition reverses the sexual dysfunction induced by the first composition.

49. The method of claim 48, wherein a decrease in the number of observed penile erections in the males receiving the composition, relative to the males receiving placebo, indicates that the composition induces sexual dysfunction in the male mammal.

50. The method of claim 48, wherein an increase in the number of observed penile erections in the males receiving the composition, relative to the males receiving placebo, indicates that the composition enhances sexual function in the male mammal.

51. The method of claim 48, wherein the composition comprises a small molecule.

52. The method of claim 48, wherein the small molecule is an antidepressant, an anxiolytic or an antipsychotic.

53. The method of claim 52, wherein the antidepressant is a serotonin reuptake inhibitor, a dopamine/norepinephrine reuptake inhibitor, a tricyclic, a norepinephrine reuptake inhibitor or any combination thereof.

54. The method of claim 51, wherein the small molecule is a compound used to treat erectile dysfunction.

55. The method of claim 48, wherein the composition is administered to the one or more male mammals daily for about 1 to about 30 days prior to placement of the one or more males in the testing arena.

56. The method of claim 48, wherein the composition is administered to the one or more male mammals daily for about 5 to about 20 days prior to placement of the one or more males in the testing arena.

57. The method of claim 48, wherein the composition is administered to the one or more male mammals daily for about 7 to about 14 days prior to placement of the one or more males in the testing arena.

58. The method of claim 48, wherein the amount of the composition is administered to the one or more male mammals daily for about 7 days.

59. The method of claim 48, wherein the amount of the composition is administered to the one or more male mammals daily for about 14 days.

60. The method of claim 48, wherein the separating means is a first containing means containing a male mammal and a second cage containing means containing the one or more female mammals.

61. The method according to claim 60, wherein the containing means is a cage.

62. The method of claim 48, wherein the separating means is a first containing means containing two or more male mammals and a second containing means containing the one or more female mammals.

63. The method according to claim 62, wherein the containing means is a cage

64. The method of claim 48, wherein the separating means is a dividing barrier placed into the testing arena, wherein a male mammal and the one or more female mammals are on opposite sides of the barrier.

65. The method according to claim 64, wherein the dividing barrier is glass or plexi-glass.

66. The method of claim 48, wherein the separating means is a dividing barrier placed into the testing arena, wherein two or more male mammals and the one or more female mammals are on opposite sides of the barrier.

67. The method according to claim 66, wherein the dividing barrier is glass or plexi-glass.

68. The method of claim 48, wherein the male and female mammals are rodents.

69. The method of claim 68, wherein the rodents are rats.

70. The method of claim 69, wherein the male rats are Sprague-Dawley rats, Wister rats, Long-Evans rats or any combination thereof.

71. The method of claim 69, wherein the female rats are Sprague-Dawley rats, Wister rats, Long-Evans rats or any combination thereof.

72. The method of claim 69, wherein the rats are about 8 to 10 weeks of age.

73. The method of claim 48, wherein the male and female mammals are non-human primates.

74. The method of claim 48, wherein about 5 to 10 female mammals are present in the testing arena.

75. The method of claim 48, wherein about 5 to 20 female mammals are present in the testing arena.

76. The method of claim 48, wherein about 1 to 100 female mammals are present in the testing arena.

77. The method of claim 48, wherein about 1 to 1000 female mammals are present in the testing arena.

78. The method of claim 48, wherein the male mammals are further defined as sexually mature males.

79. The method of claim 48, wherein the male mammals are about 8 to 10 weeks of age.

80. The method of claim 48, wherein the quantity of penile erections is measured for about a 1 to 120 minute period of time.

81. The method of claim 48, wherein the quantity of penile erections is measured for about a 1 to 90 minute period of time.

82. The method of claim 48, wherein the quantity of penile erections is measured for about a 1 to 60 minute period of time.

83. The method of claim 48, wherein the quantity of penile erections is measured for about a 1 to 30 minute period of time.

84. The method of claim 48, wherein the quantity of penile erections is measured for about a 1 to 15 minute period of time.

85. The method of claim 48, wherein the quantity of penile erections is measured during light phase of the circadian period.

86. The method of claim 48, wherein the quantity of penile erections is measured during light phase of the circadian period no earlier than 6 hours after lights on.

87. The method of claim 48, wherein the quantity of penile erections is measured during dark phase of the circadian period.

88. The method of claim 48, wherein each of the one or more male mammals are mated with one or more female mammals prior to being administered the pharmaceutical composition.

89. The method of claim 88, wherein the male mammals further mate one or more times with one or more female mammals during administration of the pharmaceutical composition.

90. The method of claim 48, wherein the testing arena is supplied with a white noise background.

91. The method of claim 88, wherein the white noise background is at about 65 dB.

92. The method of claim 48, wherein the males and females are sexually mature at the time of mating.

93. The method of claim 48, wherein the females are sexually receptive, wherein sexually receptive is defined as a natural estrous cycle.

94. The method of claim 48, wherein the females are sexually receptive, wherein sexually receptive is defined as a chemically induced estrous cycle.

95. A pharmaceutical composition in an amount sufficient to provide an antidepressant effect without causing sexual dysfunction when administered to a human subject, wherein the amount of the pharmaceutical composition is determined according to the method of claim 1.

96. The composition of claim 95, wherein the antidepressant is a serotonin reuptake inhibitor, a dopamine/norepinephrine reuptake inhibitor, a tricyclic, a norepinephrine reuptake inhibitor or any combination thereof.

97. A pharmaceutical composition in an amount sufficient to provide an anxiolytic effect without causing sexual dysfunction when administered to a human subject, wherein the amount of the pharmaceutical composition is determined according to the method of claim 1.

98. A pharmaceutical composition in an amount sufficient to provide an antipsychotic effect without causing sexual dysfunction when administered to a human subject, wherein the amount of the pharmaceutical composition is determined according to the method of claim 1.

99. A pharmaceutical composition in an amount sufficient to provide enhanced sexual function in a human subject, wherein the amount of the pharmaceutical composition is determined according to the method of claim 1.

100. A method of determining the amount of a pharmaceutical composition to administer to a patient to prevent impairment of sexual function or to improve sexual function comprising:
monitoring the quantity of penile erections in male animals that are administered the amount of the agent and then placed in a testing arena that exposes the male animals to female animals through a barrier that prevents physical contact between the male animals and any female animal while not preventing the male animals from seeing, smelling or hearing a female animal.

101. An apparatus for testing the effect of a composition on sexual function in a mammal comprising:
a separating means which prevents physical contact between the male and female mammals, wherein the separating means does not inhibit or prevent the exchange of olfactory, auditory or visual stimuli between the male and females.

102. The apparatus of claim 100, wherein the separating means is transparent on the sides and has perforations or an open space on top to permit the free flow of air and sound into and out of the solid enclosure, said solid enclosure having a sealable opening through which small animals may be placed within said solid enclosure.

103. The apparatus of claim 100, wherein the solid enclosure is plexiglas.

104. The apparatus of claim 100, wherein the solid enclosure has a depth of about twice its width and a ceiling less than its width.

105. The apparatus of claim 100, wherein the solid enclosure is about 10 inches to about 30 inches deep.

106. The apparatus of claim 100, wherein the solid enclosure is about 19 inches deep.

107. The apparatus of claim 100, wherein the solid enclosure is about 6 inches to about 15 inches wide.

108. The apparatus of claim 100, wherein the solid enclosure is about 10½ inches wide.

109. The apparatus of claim 100, wherein the solid enclosure has a ceiling of about 4 inches to about 14 inches.

110. The apparatus of claim 100, wherein the solid enclosure has a ceiling of about 8 inches.

111. The apparatus of claim 100, wherein the internal dimensions of the solid enclosure are about 10½ inches×19 inches×8 inches.

112. The apparatus of claim 100, wherein the external dimensions of the solid enclosure are about 10½ inches wide×19 inches deep×11 inches high.
